(19) United States
(12) Patent Application Publication
(10) Pub. No.: US 2010/0121198 A1
(43) Pub. Date: May 13, 2010

(54) Multi-Purpose Illumination System And Method For Tissue Examination

(75) Inventors: John West, Arroyo Grande, CA (US); Robert Ibsen, Santa Maria, CA (US)

Correspondence Address: SULLIVAN & WORCESTER LLP, 1666 K Street NW, Washington, DC, 20006, US

(73) Assignee: Den-Mat Holdings LLC, Santa Maria, CA (US)

(21) Appl. No.: 12/266,722

(22) Filed: Nov. 7, 2008

(51) Int. Cl. A61B 6/00; F21V 9/00; H05B 37/02; H05B 39/04

(52) U.S. Cl. 600/476; 362/231; 315/294; 315/158

(57) ABSTRACT

The present invention is a multi-purpose light source of a unique design and specialized attachments. The invention encompasses a system for the illumination and visual detection of normal and abnormal biologic tissue comprising a light source, a light transmitting device and a light viewing device wherein said device allows for the simultaneous illumination and viewing of target tissue.

### CROSS REFERENCE TO RELATED APPLICATIONS

This application claims the priority of U.S. Provisional Application No. 60/006,002 filed on Dec. 13, 2007, the disclosure of which is expressly incorporated by reference herein.

### FIELD OF THE INVENTION

The present invention relates generally to light sources and systems for use in dental and medical procedures.

### BACKGROUND OF THE INVENTION

There are numerous dental and medical procedures today that require a source of electromagnetic radiation. Some examples of these light producing instruments used in typical dental and medical practices include; resin light curing units, peroxide tooth whitening systems, soft and hard tissue lasers, caries detection, oral tissue examination, tooth transillumination, tooth color matching, and illumination of the oral cavity. Additional uses of light in the dental and medical practices may include bio-stimulation, pain relief, and other surgical and therapeutic indications.

Current cosmetic and surgical soft tissue procedures are performed with hand instruments like a scalpel, electrosurgical instruments, and lasers. Although instruments like a scalpel are the standard for surgical precision there is no hemostasis. Electrosurgical instruments provide hemostasis but are not as precise as hand instruments or lasers. Lasers can be used like a hand instrument to ablate, incise, excise, resect, dissect, or amputate tissue with contact fiber providing tactile feedback. Hemostasis provides clear field at the target site. Cutting with lasers is slower than hand instruments but provide better access to tissue in confined areas. Lasers have the disadvantage of being monochromatic and are expensive. The selection of wavelengths for diode lasers currently used for cosmetic and surgical soft tissue procedures is not based on the maximum absorption wavelength of target tissue. A very limited number of wavelengths are currently available with enough power to have the desired effect on tissue. High power laser diodes were primarily developed to optically pump (excite) solid-state lasers into stimulated emission. Typical wavelengths are 808 nm, 810 nm, 830 nm, and 980 nm. None of these wavelengths match the peak absorptive region of soft tissue targets such as hemoglobin, melanin, and water. Water is especially important in light-tissue interactions because it is prevalent in significant amounts in all tissue. Light energy that is absorbed by water molecules is converted to heat and provides a reliable method of heating adjacent tissue. Hemoglobin and melanin may not always be present to such a high degree to act as a heat conductor. Because the laser diodes do not closely match the peaks of targets the desired tissue interaction takes longer and/or power levels need to be increased. As power levels are increased heat transfer to non-target tissues and increased penetration depth become a concern.

Light curable composite resins have been an important part of dentistry for over 20 years. These resins are commonly used for preparing restorations, cementation of restorations, and a number of other dental restorative procedures such that light curing is now a standard procedure in dentistry.

These light curable resins used by dentists for tooth restoration and repair require a light cure unit to initiate polymerization. Initial curing lights consisted of halogen devices, first with light sources removed from the point of application and thereafter with light transmitted to the point of application through long fibers. Following that, light curing guns were introduced. These devices typically used halogen light sources with short fused fiber optic light guides close to the lamp to apply high intensity light at the point of application.

The most common way to detect disease is by visual inspection of the suspect tissue. Because the human eye is not optimized for this task. Numerous examples of research with healthy and abnormal oral tissues demonstrate that oral mucosal abnormalities can be visualized with the excitation and fluorescence of tissues with light. See, e.g., U.S. Pat. No. 6,110,106. It has been shown that the wavelengths with peaks at around 405 nm and 436 nm are favorable for the direct visualization of tissue in the oral cavity. Utzinger et al, Biomedical Engineering, Vol. 50, Issue 3, 396-399, March 2003, developed a light producing instrument that demonstrated to be effective in the identification of abnormal oral tissues.

In order to offer the benefits of these instruments, the practitioner must purchase each unit individually with most costing several thousands of dollars each. Often, when faced with financial constraints, the practitioner must decide against one or more of these instruments thereby reducing the quality of care provided to the dental patient.

There are numerous dental and medical procedures today that require a source of electromagnetic radiation. Until now, each of these procedures typically required their own, specialized source of electromagnetic radiation. The purpose of the present invention is to provide one source of electromagnetic radiation that can emulate the characteristics of one or more of a group of electromagnetic radiation generating instruments.

### SUMMARY OF THE INVENTION

The present invention is a multi-purpose light source of a unique design and specialized attachments which are also independently unique that can be used for, but not limited to, dental, medical, cosmetic, and industrial applications and procedures. Specifically the spectral irradiance of the light source can be controlled in such a way as to allow it to be used for procedures currently performed by lasers, electrosurgical devices, and hand instruments while retaining the benefits of the light source for other uses such as the photo-initiation of resins, tooth whitening, fluorescence, and illumination. The present invention may be used instead of a laser either independently or in conjunction with electrosurgical devices and hand instruments.

One embodiment of the invention encompasses a system for the illumination and visual detection of normal and abnormal biologic tissue comprising a light source, a light transmitting device and a light viewing device wherein said device allows for the simultaneous illumination and viewing of target tissue.

Another embodiment of the invention encompasses a system for the illumination and visual detection of normal and abnormal biologic tissue comprising wherein one or more illumination and viewing light wavelengths are transmitted, blocked, and attenuated by one or more filters, mirrors, and windows selectively useful for the simultaneous illumination and viewing of target tissue.

A further embodiment of the invention encompasses a system for the illumination and visual detection of normal and abnormal biologic tissue comprising a light source, a light transmitting device and a light viewing device wherein said device allows for the simultaneous illumination and viewing of target tissue and wherein said light source is at least one of a halogen light source, a xenon light source, a metal arc light source, a metal halide light source, a mercury vapor light source a sodium light source, an LED light source, an LED emitter light source, an LED die light source, an LED array light source, or a laser light source.

Another embodiment of the invention encompasses a system for the illumination and visual detection of normal and abnormal biologic tissue comprising a light source, a light transmitting device and a light viewing device wherein said device allows for the simultaneous illumination and viewing of target tissue and wherein said light source is one of a plurality of light sources.

One embodiment of the invention encompasses a system for the illumination and visual detection of normal and abnormal biologic tissue comprising a housing with a light transmitting end for transmitting light to the target tissue and a light viewing end for viewing light emitted from the target tissue.

Another embodiment of the invention encompasses a system for the illumination and visual detection of normal and abnormal biologic tissue wherein light received from the light source and light emitted from the target tissue are transmitted through one or more optical components including filters, mirrors, lens, and windows.

An embodiment of the invention encompasses a system for the illumination and visual detection of normal and abnormal biologic tissue, wherein the system is a handheld system.

A further embodiment of the invention encompasses a system for the illumination and visual detection of normal and abnormal biologic tissue wherein one or more illumination and viewing light wavelengths are transmitted, blocked, and attenuated by one or more filters, mirrors, and windows located in the transmitted or received light path in one or more places in the light source or light transmitting device.

Another embodiment of the invention encompasses a system for the illumination and visual detection of normal and abnormal biologic tissue wherein the quantity of illumination light is selectively controlled through the light source from a group of one or more steady state quantities from minimum to maximum illumination or variable quantities and light profiles such as a linear or non-linear increase over some time from minimum to maximum illumination or pulsing.

The invention further contemplates a multi-purpose light comprising a power supply that adjusts a quantity of light produced, an input through which user data, including a desired quantity of light, can be entered by the user, and a controller that communicates with the power supply to control the quantity of light produced.

Another embodiment of the instant invention contemplates a multi-purpose light comprising a power supply that adjusts a quantity of light produced, an input, through which user data, including a desired quantity of light, can be entered by the user, a light quantity regulation means including one or more light measuring device disposed in the illumination path that measures a quantity of light produced, a controller that together with the measuring device forms at least part of a feedback loop, the controller communicating with the power supply and measuring device and receiving user data, wherein said power supply adjusts a quantity of light emitted by the light source based on communication provided by the controller so that the quantity of light received for the light source becomes substantially equal to said desired quantity of light.

The instant invention also contemplates multi-purpose light comprising a measuring device selected from a photodiode, a photodetector, a phototransistor, a photo resistor, a light-to-analog light sensor, a light-to-digital light sensor, a light-to-frequency light sensor, and combinations thereof.

### BRIEF DESCRIPTION OF THE FIGURES

FIG. 1 shows an overview of an illumination system in accordance with the instant invention.

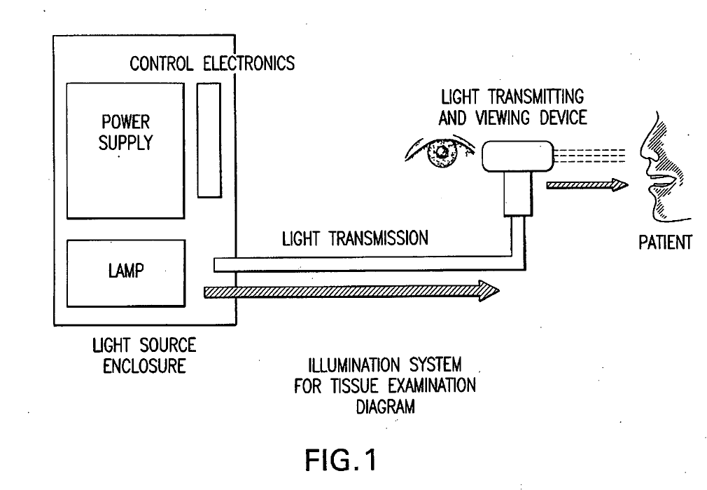

FIG. 2 shows detail of light transmitting and viewing devices for using in the illumination system of the instant invention.

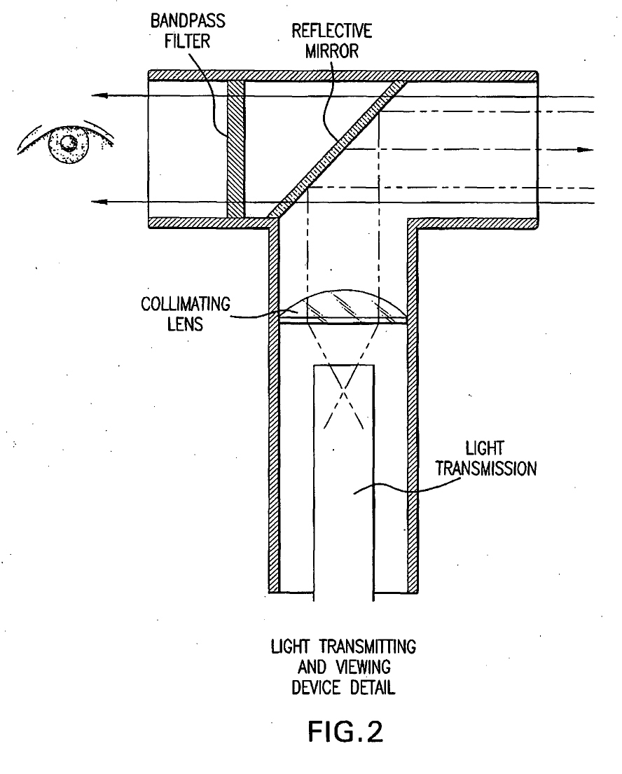

FIG. 3 shows the spectral output and excitation wavelengths of an oral tissue examination instrument.

FIG. 4 shows the broad spectral output of an illumination system in accordance with the present invention before modification of the spectral output.

FIG. 5 shows the spectral output of an illumination system in accordance with the present invention after modulation has occurred.

FIG. 6 shows the broad spectrum of the light source of the present invention.

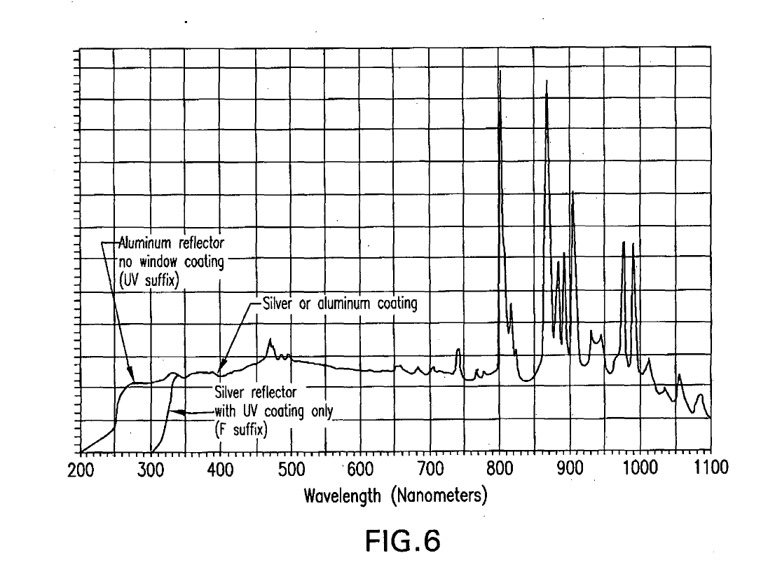

FIG. 7 shows the absorptive regions of different biological tissues and organic matter.

### DETAILED DESCRIPTION OF THE INVENTION

For simplicity and illustrative purposes, the principles of the present invention are described by referring to various exemplary embodiments thereof. Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention is a multi-purpose light source of a unique design and specialized attachments which are also independently unique that can be used for, but not limited to, dental, medical, cosmetic, and industrial applications and procedures. Specifically the spectral irradiance of the light source can be controlled in such a way as to allow it to be used for procedures currently performed by lasers, electrosurgical devices, and hand instruments while retaining the benefits of the light source for other uses such as the photo-initiation of resins, tooth whitening, fluorescence, and illumination. The present invention may be used instead of a laser either independently or in conjunction with electrosurgical devices and hand instruments.

The present invention generates electromagnetic energy that is controlled and delivered by unique optical, electronic, and electro-mechanical devices for the purpose of producing predictable effects on biological tissue, photo initiation of dental light cure resins and other light cure materials, activation of tooth whitening agents, and illumination. Indications of use are for, but not limited to, Dental, Medical, Cosmetic, and Industrial applications and procedures.

The present invention may be used in dental and medical procedures where light interaction with biological tissue through an optical component less than 1 mm in diameter or larger is desired. The present invention may be used for the photo initiation of dental light cure resins or other light cure materials through an optical component less than 1 mm in diameter or larger. The present invention may be used to activate tooth whitening agents through an optical component on both upper and lower tooth arches simultaneously or one tooth at a time. The present invention may be used as a source of illumination in the oral cavity, in medical surgery, for machine vision, or other.

Tissue Interactions Include Indications For Use In:
Open and Endoscopic Surgery; light assisted procedures provide a level of surgical precision not available with other mechanical means and where the benefits of the hemostasis effect of the light is realized.
Photodynamic Therapy and Biostimulation; relatively low light levels are used to alter or otherwise stimulate living tissue in therapeutically useful ways.
Pain Control; light induced analgesia and nerve stimulation therapy.
Tissue welding and fusion; seals biological tissue without sutures.

Primary Light-Tissue Interactions:
Photothermal where light is absorbed by tissue and converted to heat energy or where water or other molecules absorb light energy and heat tissues indirectly.

Other Light-Tissue Interactions:
Photochemical/Photodynamic; light absorbing molecules result in a chemical reaction with tissue or the formulation of a biochemically reactive singlet oxygen molecule.
Biostimulation employs relatively low light levels to stimulate healing of tissue and pain relief.

The present invention is of unique construction that may allow the delivery of appropriate levels of electromagnetic energy to target tissue through an optical component less than 1 mm in diameter or larger while still retaining the benefits of the light source for the photo initiation of dental light cure resins or other light cure materials, the activation of tooth whitening agents, and as a general illumination source.

Delivery of light energy through an optical component less than 1 mm in diameter or larger is necessary for surgical precision on target tissue. This is especially necessary for procedures within the periodontal pocket and other confined areas. The ability to deliver appropriate levels of electromagnetic energy to target tissue through an optical component less than 1 mm in diameter or larger while still retaining the benefit of the light source for other uses is made possible through the unique design of optical and other component designs contained in the light source. The light source, in total, and the unique components individually make up the present invention.

Individual components that comprise the present invention include but are not limited to:
1) xenon lamp with unique reflector geometry and arc gap
2) light guide and hand piece with optical taper and focusing optics
3) micro-taper tip less than 1 mm diameter
4) filter changer with Visible and IR band pass filters
5) variable switching power supply with self-monitoring feedback loop

### EXAMPLE 1

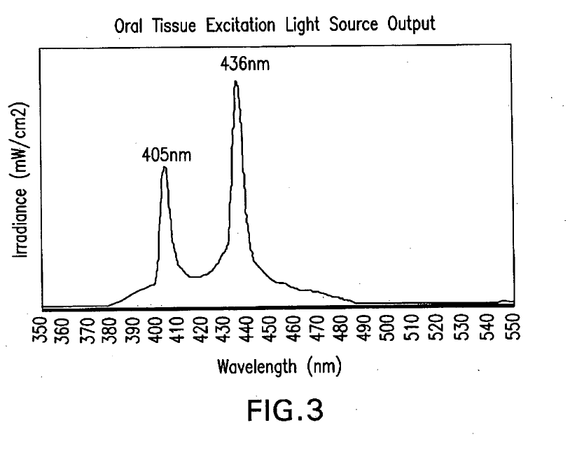

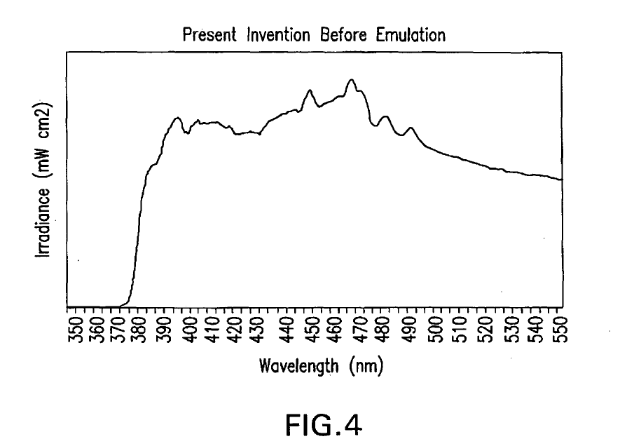

A light producing instrument was developed and demonstrated in clinical trials to be highly effective in the identification of abnormal oral tissues. The spectral irradiance of this instrument is shown in FIG. 3. FIG. 3 shows the spectral output and excitation wavelengths of the Oral Tissue Examination Instrument with peak wavelengths of 405 nm and 436 nm (nanometers). FIG. 4 shows the broad spectral output of the present invention before modification to produce the output required for tissue examination. The light source of the present invention produces a wide range of wavelengths including wavelengths of 405 nm and 436 nm (nanometers). In order to wavelengths of 405 nm and 436 nm it is necessary for the spectral output of the present invention to be filtered.

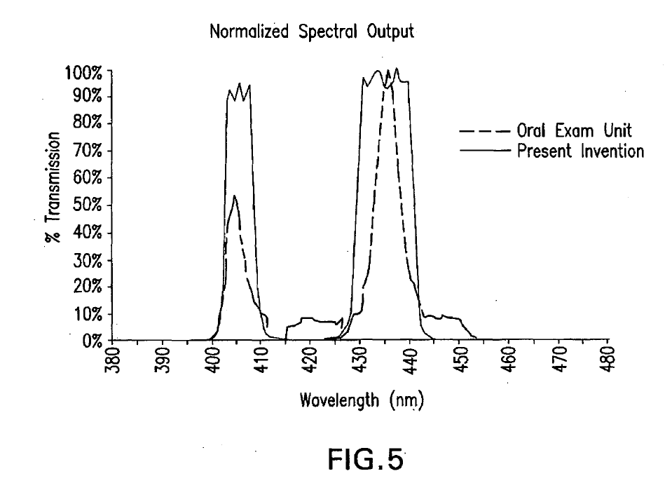

FIG. 5 shows the present invention after emulation has occurred. Optical filters along with Real Time Optical Regulation are used to emulate the spectral irradiance of the Oral Tissue Examination Instrument so that the output waveforms are substantially the same.

One advantage of the present invention is that it can be used for multiple indications. When not in use as an Oral Tissue Examination Instrument the optical filters, real time optical regulation and other means can be changed to emulate the other dental instruments included in the group listed.

The broad spectrum light of the present invention has a range of wavelengths similar to those shown in FIG. 4 including UV, Visible, and IR.

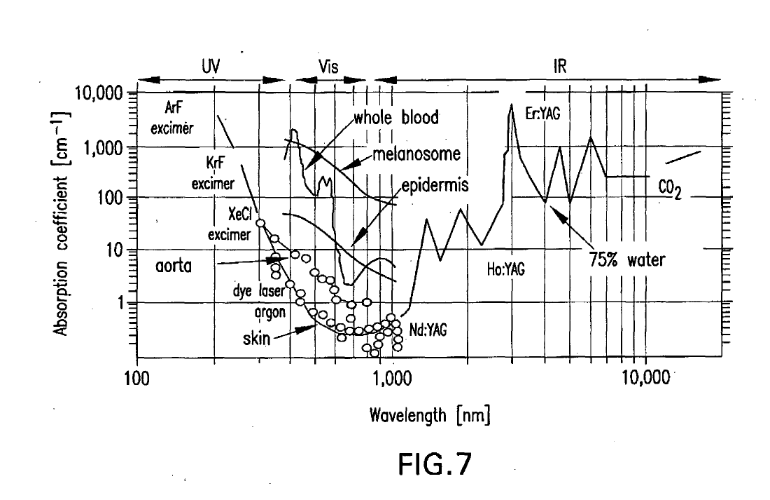

The absorptive region of different biologic tissues and organic matter is established in research and literature as show in FIG. 7. By emulating the known absorptive wavelengths and understanding the interactions of electromagnetic energy with matter it is possible to predict the results.

The present invention has the potential to be used for a wide variety of indications in the dental and health fields. The only current alternative is the use of many individual procedure instruments and the associated high cost of procurement and maintenance.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of devices would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed is:

1. A system for the illumination and visual detection of normal and abnormal biologic tissue comprising:
at least one light source;
a light transmitting and viewing device;
wherein said device allows for the simultaneous illumination and viewing of target tissue; and
wherein one or more light wavelengths are transmitted, blocked, or attenuated by one or more filters.

2. The system of claim 1 wherein said light source is selected from the group of: a halogen light source, a xenon light source, a metal arc light source, a metal halide light source, a mercury vapor light source, a sodium light source, an LED light source, an LED emitter light source, an LED die light source, an LED array light source, and a laser light source.

3. The system of claim 2 wherein the system comprises a plurality of light sources.

4. The light transmitting device of claim 1 comprising of a housing with a light transmitting end for transmitting light to the target tissue and a light viewing end for viewing light emitted from the target tissue.

5. The device of claim 4 transmitting light received from the light source and light emitted from the target tissue through one or more optical components including filters, mirrors, lens, and windows.

6. The system of claim 1 wherein the light source is powered by alternating current.

7. The system of claim 1 wherein the light source is powered by direct current.

8. A hand held illumination and visual detection system according to claim 1.

9. A cordless hand held illumination and visual detection system according to claim 1.

10. The cordless hand held system of claim 9 wherein the system is powered by direct current is attached to an alternating current/direct current converter or is attached to a direct current/alternating current converter.

11. The system of claim 1 wherein the one or more illumination and viewing light wavelengths are transmitted, blocked, and attenuated by one or more filters, mirrors, and windows located in the transmitted or received light path in one or more places in the light source or light transmitting device.

12. The system of claim 1 wherein the quantity of illumination light is selectively controlled through the light source from a group of one or more steady state quantities from minimum to maximum illumination or variable quantities and light profiles such as a linear or non-linear increase over some time from minimum to maximum illumination or pulsing.

13. The light source of claim 1 comprising:
a power supply that adjusts a quantity of light produced.

14. The light source of claim 1 comprising:
a power supply that adjusts a quantity of light produced.
an input, through which user data, including a desired quantity of light, can be entered by the user.
a controller that communicates with the power supply to control the quantity of light produced.

15. The light source of claim 1 comprising:
a power supply that adjusts a quantity of light produced;
an input, through which user data, including a desired quantity of light, can be entered by the user;
a light quantity regulation means including;
one or more light measuring device disposed in the illumination path that measures a quantity of light produced;
a controller that together with the measuring device forms at least part of a feedback loop, the controller communicating with the power supply and measuring device and receiving user data;
wherein said power supply adjusts a quantity of light emitted by the light source based on communication provided by the controller so that the quantity of light received for the light source becomes substantially equal to said desired quantity of light.

16. The system of claim 15 where said measuring device is selected from a photodiode, a photodetector, a phototransistor, a photo resistor, a light-to-analog light sensor, a light-to-digital light sensor, a light-to-frequency light sensor, and combinations thereof.

17. The system of claim 15 wherein said measuring device is one of a plurality of measuring devices.

18. The system of claim 15 wherein the controller includes at least one control device adapted to receive input from the light measuring device, perform a comparison of the input received from the light measuring device and a set point, and adjust power supplied to the light source based on the comparison.

19. The system of claim 15 wherein the set point is a quantity of light.

20. The system of claim 15 wherein the control device is a microprocessor or a microcontroller.

21. The system of claim 15 wherein the controller communicates with the power supply so that said quantity of light emitted by the light source is adjusted by analog voltage regulation, analog current regulation, pulse width modulated voltage, pulse width modulated current, or a digital signal.

22. A method for the illumination and visual detection of normal and abnormal biologic tissue using a real time optically regulated examination unit comprising;
supplying a quantity of light be way of a light source;
receiving data, including a desired light quantity, entered by a user;
measuring a light quantity received from the light source;
performing a comparison of the desired light quantity with the light quantity received from the light source; and
adjusting the quantity of light supplied by way of the light source based on said comparison.

23. The method of claim 22 wherein the method is a method of examination of normal and abnormal biologic tissue where a quantity of light is supplied to said tissue.

24. The method of claim 22 wherein the method is a method of examination of normal and abnormal biologic tissue where a quantity of light is emitted from said tissue.

25. The method of claim 22 wherein the desired quantity of regulated light is used to compensate for shadows on the tissue.

26. A method for the illumination and visual detection of normal and abnormal biologic tissue comprising;
transmitting one or more illumination light wavelengths through one or more optical components including filters, mirrors, lens, and windows to the target tissue with one or more steady state or variable quantities of light.
viewing one or more light wavelengths emitted from the target tissue through one or more optical components including filters, mirrors, lens, and windows with one or more steady state or variable quantities of light.
